(19) United States
(12) Patent Application Publication
(10) Pub. No.: US 2008/0014251 A1
(43) Pub. Date: Jan. 17, 2008

(54) Hemostatic compound and its use

(75) Inventors: Philip Benz, Portland, OR (US); Herbert Semler, Portland, OR (US)

Correspondence Address: ATER WYNNE LLP, 222 SW COLUMBIA, SUITE 1800, PORTLAND, OR, 97201-6618, US

(73) Assignee: Advanced Vascular Dynamics

(21) Appl. No.: 11/486,780

(22) Filed: Jul. 14, 2006

(51) Int. Cl. A61L 15/00; A61K 36/886

(52) U.S. Cl. 424/445; 424/744

(57) ABSTRACT

A novel hemostatic compound includes a hemostatic agent and a transdermal migration-enhancing agent, the agents combined in a defined ratio and in a form capable of topical application to an abrasion, puncture or incision site and capable at the site of inhibiting bleeding. In accordance with one embodiment of the invention, the hemostatic agent is a vasoconstrictor and/or a procoagulant, and the agents are combined in a liquid or semi-liquid form. A compound-gelling agent and/or an evaporative agent and/or an evaporative gel can be combined with the hemostatic and migration-enhancing agents in a defined ratio. In accordance with another embodiment of the invention, the invented hemostatic compound includes a hemostatic agent and a compound-gelling agent, the latter being more particularly an evaporative gel. Other components such as sterilizing, analgesic or antibacterial agents can be added to the various invented compounds. A method for the compound's use during a cannulation procedure or as first aid also is disclosed.

### BACKGROUND OF THE INVENTION

This invention relates generally to the field of medicine. More particularly, it concerns sanitizing a wound or abrasion and retarding blood flow thereat.

Catheterization or other cannulation procedures conventionally require the application of pressure to the region of a mammalian, e.g. human, body surrounding the blood vessel from which a catheterization sheath is withdrawn. Such pressure attempts to inhibit bleeding from the catheterized blood vessel. Pressure conventionally is applied manually, e.g. by use of the clinician's fingers and/or hand. Often, compression devices are used to assist in leveraging and focusing the compression on the blood vessel. One such compression device is the Compressar® apparatus, which uses interchangeable pads and is configured to concentrate pressure in a desired region around the blood vessel. Compressar® is a registered trademark of Advanced Vascular Dynamics of Portland, Oreg., USA, world-wide rights reserved.

A more recent advance is described and illustrated in co-pending design patent application entitled HANDHELD VASCULAR COMPRESSION DEVICE, U.S. patent application Ser. No. 29/258,514, filed Apr. 20, 2006, which design application is subject to co-ownership herewith by Advanced Vascular Dynamics. A compression device incorporating that design, marketed by Advanced Vascular Dynamics under the Compass™ trademark, is cupped fittingly in the palm of the hand and uses interchangeable pads for compressing a blood vessel while removing a catheterization sheath therefrom.

Injury to tissue causes a physiological response called the clotting cascade, which can simply be described as a complex sequential interplay of many blood components which ultimately result in the formation of a stable clot at the injury site. The tissue injury triggers a sequence of chemical activity that cascades through a sequential activation and combination of circulating precursor blood proteins and substances, many of which are known as “factors”, e.g. Factor I is fibrinogen, Factor II is prothrombin, and so on. The sequence of activity in its sequential phases generally involves: a) platelet activation and aggregation at the injury site, which forms a loose platelet plug by binding to collagen exposed following endothelial rupture of blood vessels; b) substances in the blood, including factors, phospholipids, and calcium ions interact with each other to activate additional factors in the cascade, specifically thrombin from prothrombin; c) thrombin in turn converts fibrinogen into fibrin monomers, which start to form a framework within which the clot is formed; and d) fibrin monomers, combined with another converted blood factor, XIIIa, are in turn converted into fibrin polymers, which creates a strong cross-linked fibrin meshwork forming the clot.

From the above description of the mammalian body's natural biological response to puncture, incision, abrasion or other injury, it will be understood that blood clotting takes time and in the case of severe bleeding puts the body at risk of excessive loss of blood. This is especially critical in a hospital setting where a patient already may be in a weakened and vulnerable condition from catheterization, for example, of a major artery such as the femoral artery.

Conventional approaches to inhibiting bleeding involve the direct application of pressure to the catheterization site. They are labor-intensive, requiring significant care, strength and endurance on the part of the clinician, e.g. physician, technician or nurse. For example, the procedure often is repeated 1-5 times per day for twenty minutes or more by a practitioner in a modestly busy hospital or clinic, which can lead to carpal tunnel syndrome and workers' compensation or disability claims. Moreover, all are subject to the incidental application of too little or too much pressure onto the blood vessel so that the blood vessel is not fully occluded as desired to inhibit bleeding or so that the inner wall of the blood vessel is unduly pressed against other tissue or bone features. Such manual methods often produce much discomfort for catheterization patients and clinicians.

Bandages sterilizing compounds are conventionally used in first aid applications. Bandages are for absorption and/or containment of blood but do little or nothing to inhibit bleeding. Antiseptics for cleaning and/or sterilizing wounds include rubbing alcohol, iodine, betadine, chlorhexidine, and hydrogen peroxide but likewise they do little or nothing by themselves to inhibit bleeding. Spray fixatives do little more than coat the wound to protect it from particulate contamination from continued exposure. Some topical dressings containing antibiotics help fight bacterial infection when applied to a wound site. Liquid sutures, which notoriously fail to close deep or long incisions, are at best a stop gap on the way to stitches.

Vasoconstrictors and procoagulants have been used in medical and dental applications involving invasive procedures such as surgery or catheterization. Known uses are invasive, i.e. the use of such is limited to injections or surgical applications that penetrate the subject's skin or are placed directly onto internal tissue during a procedure to affect local subdermal or internal tissue and/or blood vessels. For example, epinephrine has been injected at sites, e.g. for hemostasis during and after oral surgery or drilling, or during skin surgery, e.g. skin surface repair or tissue excising (e.g. “lumpectomies” or “bumpectomies”).

Epinephrine, also known as adrenaline, is a substance naturally produced by the adrenal glands, and which has also been synthesized. Possessing complex target organ effects when administered systemically, epinephrine is a potent agonist at both alpha- and beta-receptors. A nonselective adrenergic agonist; it stimulates alpha-1, alpha-2, beta-1, and beta-2 adrenergic receptors, although the degree of stimulation at these receptors varies depending on dose. Stimulation of alpha-1 receptors and post-synaptic alpha-2 receptors by epinephrine leads to arteriolar vasoconstriction. When used internally, for example, subcutaneously, as by injection or on mucosal surfaces within, for example, a nasal cavity, epinephrine constricts arterioles, thus producing local vasoconstriction and hemostasis in small blood vessels. For purposes of the present invention the term vasoconstrictor includes compounds including such vasoconstrictors, for example, epinephrine, in solid, liquid and semi-liquid form.

(The major therapeutic effects of systemic epinephrine include: bronchial smooth muscle relaxation, cardiac stimulation, vasodilation in skeletal muscle, and stimulation of glycogenolysis in the liver and other calorigenic mechanisms. The potent cardiac effects of epinephrine are primarily mediated via stimulation of beta-1 receptors on the myocardium and conduction system of the heart. The stimulation of these receptors results in both increased inotropic and chronotropic effects. Systolic blood pressure is usually elevated as a result of increased inotropy, although diastolic blood pressure is decreased secondary to epinephrine-induced vasodilation. Epinephrine indirectly causes coronary artery vasodilation, particularly during cardiac arrest. Increased myocardial excitability and automaticity markedly increase the potential for developing dysrhythmias. The effects of systemic epinephrine are not germane to the present invention. This is because of the unique external application and delivery modality of the invented compound and its use, as described and illustrated herein.)

Thrombin and fibrin preparations have also been used for hemostasis in surgical procedures and in vascular catheterization procedures. Chitosan and acetylglucosamine (including poly-.beta.-1.fwdarw.4-N-acetylglucosamine polysaccharide species—see U.S. Pat. No. 6,630,459) preparations, all in solid form, have been used in commercially-available topical applications for post-catheterization vascular hemostasis as well as in other hemostatic applications, including deployment on severe injuries. Mineral preparations, including zeolite, and preparations derived from potato starches have likewise found use in solid form in hemostatic devices applied to severe injuries involving copious bleeding.

Chitosans are cationic polysaccharides having a wide range of biomedical applications and generally possessing biocompatibility, biodegradability, bioadhesive and antimicrobial characteristics, in addition to their procoagulative effects. They are often derived from the chitin in sea crustacean shells and commercially available as solids, powders, and in soluble liquid form. For purposes of the present invention the term chitosan shall be considered to include chitosan, chitosan salts, for example, chlorides or hydrochlorides, and in liquid or semi-liquid forms, for example in aqueous solutions or suspensions or in a dilute acetic acid formulation. Having documented procoagulant properties which are well-known to those skilled in the art, they are commonly used for hemostasis in medical settings, and find use as topically applied solid materials, for example the product marketed under the trade name CHITOSEAL™ (available from Abbott Labs). An example of a product composed of an acetylglucosamine polysaccharide is marketed under the trade name, SYVEK™ (Marine Polymers), and is also topically applied for the purpose of achieving post-cannulation femoral hemostasis as a solid material. Another solid chitosan product known for its use in military and trauma applications is called the HEMCON™ BANDAGE (available from HemCon).

Transdermal enhancers, which may also be known as skin permeability enhancers, enable greater transport of substances, including drugs, through the skin. FIG. 1 shows a mammalian skin layer 10 in fragmentary cross section under compression from a prior art manual compression device known under the Compass™ trademark. Those of skill in the art will appreciate that FIG. 1 illustrates the skin layers and manual compression thereof in accordance with prior art techniques and devices. Those of skill also will appreciate that FIG. 1 is not drawn to scale, for the sake of clarity, and it will be understood that the skin depth is greatly exaggerated relative to the size of the human hand and compression device, and also that some of the skin layer thickness and spacing dimensions are exaggerated as well. As will be seen, use of the invented compound in connection with a cannulation procedure, e.g. a femoral artery catheterization, is contemplated as a part of the invented method for the compound's use.

As may be seen in FIG. 1, mammalian skin 10 comprises several layers, illustrated in fragmentary cross section. Those having relevance to the present invention include the topmost epidermal layer known as the stratum corneum 12, and the ordered layers lying immediately below it called, respectively, the viable epidermis 14, the dermis 16 and subcutaneous tissue 18. Once a drug passes through the relatively thin stratum corneum 12, it enters the layer of viable epidermis 14, which is more hydrated than stratum corneum 12. Below viable epidermis 14 lies dermis 16, which contains the microcirculation system 20 (smaller blood vessels), along with sweat glands 22 and hair follicles 24. Subcutaneous tissue 18, which contains major blood vessels such as a femoral artery 26, lies below dermis 16.

Stratum corneum 12, constructed of dead cells known as corneocytes connected by desmosomes and embedded in a lipid matrix, forms a formidable permeability barrier to substances of all kinds and poses the greatest obstacle to transporting substances through the skin layers. Microcirculation system 20 and subcutaneous tissue 18 are the targets of most transdermal enhancers, with respect to prior art drug delivery intended for systemic absorption/circulation into a patient's blood stream. Unlike prior art drug delivery intended for systemic absorption/circulation, the topical compound that is the subject of the present invention is dose-and-delivery controlled in order to avoid such absorption/circulation and systemic (drug) effects. Instead, the invented compound dose is delivered through the skin layers including stratum corneum 12 and viable epidermis 14 to dermis 16 to provide only a localized effect on microcirculation system 20 including the smaller blood vessels, thereby to inhibit bleeding. A cannular sheath, or cannula, 28 is shown in FIG. 1 to illustrate the method of the present invention that can optionally include manual compression (whether unassisted or assisted by the illustrated Compass™ compression device) of a patient's femoral artery during a catheterization procedure.

Various chemical methods for achieving such enhanced transport through the skin have been used and are well known to those skilled in the art. These include, but are not limited to water; glycols; oleic and linoleic acids; solvents including alkylmethyl sulfoxides, polyols, and alcohols; glycerins; isopropyl myristate; terpenes; and essential oils derived from plant materials. These substances have the effect of enabling substances, typically drugs, to cross the skin barrier, specifically the stratum corneum, to underlying skin layers or more typically to the circulatory system to so-called “systemic” effect. This transport occurs via some combination of intracellular or intercellular routes, the combination dictated by the specific substance or combination of substances employed. Drug transport via the hair follicle route or sweat gland route may also be used. But, due to their very small proportions of epidermal surface area, the routes through the stratum corneum are preferred.

Notoriously, prior art transdermal agents used, for example, in systemic drug delivery are relatively inefficient. As a result, extremely large doses of the drug are required to effect delivery of even a small dose to the intended subcutaneous site. Thus, controlling the systemic delivery of such drugs in therapeutic doses, in accordance with prior art drug delivery methods and devices, remains problematic.

Other methods of transdermal transport are also well-known, including phonophoresis and electrostimulation including iontophoresis and electroporation, however, these methods are not germane to the present invention.

The use of vasoconstrictors or procoagulants in liquid or semi-liquid or gel preparations for topical (exodermic) applications to achieve hemostasis of a puncture, incision, abrasion or other wound heretofore is unknown. Moreover, the use of transdermal enhancers as enablers in the transdermal delivery of hemostatic agents such as vasoconstrictors or procoagulants, is unknown.

### SUMMARY OF THE INVENTION

A novel hemostatic compound includes a hemostatic agent and a transdermal migration-enhancing agent, the agents combined in a defined ratio and in a form capable of topical application to an abrasion, puncture or incision site and capable at the site of inhibiting bleeding. In accordance with one embodiment of the invention, the hemostatic agent is a vasoconstrictor and/or a procoagulant, and the agents are combined in a liquid or semi-liquid form. A compound-gelling agent and/or an evaporative agent and/or an evaporative gel can be combined with the hemostatic and migration-enhancing agents in a defined ratio. In accordance with another embodiment of the invention, the invented hemostatic compound includes a hemostatic agent and a compound-gelling agent, the latter being more particularly an evaporative gel. Other components such as sterilizing, analgesic or antibacterial agents can be added to the various invented compounds. A method for the compound's use during a cannulation procedure or as first aid also is disclosed.

### BRIEF DESCRIPTION OF THE DRAWINGS

FIG. 1 is a cross-sectional elevation of mammalian skin, illustrating for background purposes the barrier layers to effective hemostatic agent delivery to a wound site (not shown).

FIG. 2 is a flowchart of the invented method in accordance with one embodiment of the invention.

FIG. 3A is a cross-sectional elevation of the use of the invented hemostatic compound in an earlier phase of its use with the catheterization sheath inserted into a subcutaneous femoral artery, in connection with inhibiting bleeding from the withdrawal of a catheterization shield from a blood vessel of a mammalian body, in accordance with one embodiment of the invention.

FIG. 3B is a cross-sectional elevation of the use of the invented hemostatic compound in a later phase of its use after the catheterization sheath has been removed from the femoral artery before the commencement of evaporation of fluid from the skin's surface and during migration of the hemostatic agent inwardly and somewhat laterally therebeneath, also in accordance with one embodiment of the invention.

### DETAILED DESCRIPTION OF THE PREFERRED EMBODIMENTS

The following definitions of agents and other terms apply herein. Those of skill in the art will appreciate that the definitions are intended to illustrate the use of the terms, but not to limit the invention. In general, the agent definitions embrace the agent's primary purpose, although it will be appreciated that the agent may produce secondary effects.

Hemostasis: stoppage of bleeding or hemorrhage.

Hemostat or hemostatic agent: a substance that substantially stops bleeding or hemorrhage.

Topical: placement on an exterior body surface.

Antiseptic or antiseptic agent: inhibits growth or reproduction of bacteria.

Sanitizer or sanitizing agent: cleans but does not necessarily sterilize.

Disinfectant or disinfecting agent: destroys, neutralizes or inhibits growth of disease-carrying organisms such as microbes.

Antibacterial or antibacterial agent: inhibits bacterial growth.

Antibiotic or antibiotic agent: a microorganism that destroys or inhibits the growth of other microorganisms.

Bactericide or bactericidal agent: kills some or all bacteria.

Astringent or astringent agent: causes non-selective tissue shrinkage.

Sterilizer or sterilizing agent: kills a very high proportion of or all bacteria.

Microbicide or microbicidal agent: kills some or all microbes.

Solid: a phase or condition that is neither gas nor liquid nor semi-liquid; characterized as being of a fixed morphology or shape incapable of flowing despite outside action; can assume a dry powder form.

Liquid or semi-liquid: a phase or condition between solid and gas; characterized as being capable at least of very slowly changing shape and being capable at most of freely flowing without outside action; amorphous.

Gel: a jelly-like substance which can be formed as a viscoelastic substance formed from coagulation of a colloidal liquid. A semi-liquid.

Evaporative gel: a gel that substantially evaporates, leaving behind some or no residue, within a defined and relatively short, period of time.

Gelling agent: assists in forming a gel.

Evaporative gelling agent: a gelling agent that assists in forming an evaporative gel.

Evaporative agent: an agent that substantially evaporates within a relatively short period of time.

Skin penetration enhancer or transdermal migration-enhancing agent: substance that enables or facilitates other substances to move through the skin.

Defined ratio: a ratio involving non-zero, i.e. at least trace, amounts of each of two or more combined constituents. A ratio that is not arbitrary.

Thus, alcohol is an example of a sanitizer with bactericidal properties; vasoconstrictors and procoagulants are examples of hemostats or hemostatic agents; betadine and chlorhexidine are examples of microbicides; Purell™ is an example of an evaporative gel. Water, isopropyl myristate, glycols and solvents including alcohols are examples of a skin penetration enhancer or transdermal migration-enhancing agent. Carbomer™ 940, an acrylic acid polymer, is an example of a gelling agent, as are members of the Carbopol™ family of rheology modifiers.

Catecholamines include epinephrine and norepinephrine. Drugs having sympathomimetic properties, including that of vasoconstriction, include phenylephrine, oxymetazoline and pseudoephedrine, and may be referred to herein as vasoconstrictors. Hematologic substances that trigger or are a part of the clotting cascade (described above) that leads to formation of a clot include fibrinogen, prothrombin, clotting “factors” (e.g. factor VIII), platelets, fibrin and thrombin. Other substances that can initiate or assist in the clotting cascade include cotton, collagen and tannic acid, as well as chitosan and acetylglucosamines (specifically including poly-.beta.-1.fwdarw.4-N-acetylglucosamine polysaccharide species—see U.S. Pat. No. 6,630,459), and, finally, minerals including zeolite, and starches derived from potato. For purposes of the present invention, such foregoing substances may include compounds thereof, in solid, liquid or semi-liquid form.

Hematologic substances and other substances that can initiate or assist in the clotting cascade may be referred to herein as procoagulants.

The invention in accordance with various preferred embodiments involves providing a liquid or semi-liquid topically-applicable compound containing a vasoconstrictor and/or a procoagulant, an evaporative gel or transdermal migration-enhancer and, optionally, one or more additional components. A vasoconstrictor or procoagulant applied at the external skin surface migrates transdermally by action of the migration-enhancer, to a subdermal wound site where it promotes blood clotting therein through vasoconstrictive and/or procoagulative action around traumatized (e.g. pierced, torn, vivisected, ruptured or otherwise opened or ulcerated) blood vessels.

A preferred embodiment of the present invention may include its formulation as an evaporative gel, whereby on application, by rubbing onto a body surface, much or substantially all of the volume of the gel evaporates within a short period of time from onset of application, leaving behind either some or no residue. The gel is a delivery mechanism for the active agent, whether vasoconstrictor, procoagulant or both; once the evaporative agent has substantially evaporated the active agents remain either on the skin surface or migrate through the surface and/or injury to the subdermal injury site. In a test series of embodiments of the present invention a one milliliter amount of the invented compound evaporated when rubbed onto skin in less than 60 seconds from commencement of application. Generally, the preferred embodiments have an evaporation time of between 10 seconds and 300 seconds in one milliliter amounts.

Depending upon the constituents of a specific embodiment it may be expected that some proportion of the volume of the compound might remain following evaporation. For example, where a generally non-soluble solid is included in the formulation, it is expected that the volume of the post-evaporation residue will be approximately equal to the volume of such non-soluble solids.

Examples of gelling agents used in an evaporative gel include high molecular weight cross-linked polymers of acrylic acid, such as members of the family of rheology modifiers known under the trade name Carbopol™. More particularly, a substance commercially known as Carbomer™ 940, can be used. For purposes specific to this embodiment of the present invention, Carbomer™ 940 would be mixed directly into an evaporative agent, for example, an alcohol, more particularly ethanol at a strength of 195 proof, instead of into water, to create the evaporative gel. The other substances required or desired for the compound also can be mixed into the gel formulation. The rapid evaporation of the gel is caused by the large volume proportion of an alcohol or other evaporative agent contained therein.

Tests of an early version of the present invention indicated a need for a viscosity, or resistance to flow, greater than experienced with most liquids and gels, specifically those gels having sanitizer properties, for example the gel sold under the PURELL® brand. A common metric of viscosity is the centipoise (equal to one millipascal second), where, at room temperature, i.e. approximately 70 degrees Fahrenheit (70° F.), water has a viscosity of one centipoise, blood might have a viscosity of approximately 10 centipoise, molasses might have a viscosity of approximately 5,000 centipoise, tomato paste might have a value of approximately 150,000 centipoise, and so on.

A problem identified with a compound having a low viscosity, for example generally between one and 100 centipoise, is that the components in the compound will quickly spread over an area larger than the target area of application because of its tendency to flow freely, resulting in a lower-than-desired density of the compound remaining on the target area for a shorter-than-desired period of time. As viscosity increases, the compound flows less freely, thereby permitting the compound to remain on the target area in sufficient density for a longer period of time. Subsequent testing of an embodiment has shown a compound having a viscosity of greater than approximately 25,000 centipoise but less than approximately 250,000 centipoise to have desirable viscosity characteristics. The viscosity of the compound can be adjusted by varying the proportion of gelling agent and/or liquids or semi-liquids used.

These viscosity parameters, as well as other parameters set forth herein, are illustrative and are not intended to limit the claims unless the claims themselves are expressly so limited.

The following examples of preferred embodiments are generally for topical application to a patient following a cannulation procedure, more particularly a femoral catheterization procedure at the time the cannula is withdrawn from the patient's femoral artery. The amount of the hemostatic compound, including an evaporative gel or migration-enhancing agent (and other optionally added components), to be applied is between approximately 0.1 milliliters and approximately 10 milliliters, more particularly in the range of approximately 0.25 milliliters and approximately 3.0 milliliters.

### EXAMPLE COMPOUNDS

### Example 1

A hemostatic compound includes the following substances: a vasoconstrictor, a sanitizer, a gelling agent, and a transdermal-enhancing agent. More particularly, this compound includes: epinephrine in a concentration by volume of between approximately 0.1% and approximately 40%, or more particularly between approximately 5% and approximately 20%; ethanol in a concentration by volume of between approximately 20% and approximately 90%, or more particularly between approximately 40% and approximately 80%; Carbomer™ 940 in a concentration by volume of between approximately 0.1% and approximately 5%, or more particularly between approximately 0.25% and approximately 1.0%; isopropyl myristate in a concentration by volume of between approximately 1% and approximately 15%, or more particularly between approximately 3% and approximately 10%.

### Example 2

A hemostatic compound includes the following substances: a vasoconstrictor, a sanitizer, a gelling agent, and a transdermal-enhancing agent. This compound further includes, in addition to those substances described in Example 1 herein, a disinfectant having a microbicidal property which persists as a microbicidally effective residue following its application. More particularly, the compound of Example 1 further includes chlorhexidine gluconate in a concentration of between approximately 0.5% and approximately 20%, or more particularly between approximately 1% and approximately 5%.

### Example 3

A hemostatic compound includes the following substances: a procoagulant, a sanitizer, a gelling agent, and a transdermal-enhancing agent. More particularly, this compound includes: chitosan in a concentration by volume of between approximately 2% and approximately 50%, or more particularly between approximately 10% and approximately 40%; ethanol in a concentration by volume of between approximately 20% and approximately 90%, or more particularly between approximately 40% and approximately 80%; Carbomer™ 940 in a concentration by volume of between approximately 0.1% and approximately 5%, or more particularly between approximately 0.25% and approximately 1.0%; isopropyl myristate in a concentration by volume of between approximately 1% and approximately 15%, or more particularly between approximately 3% and approximately 10%.

Other examples and embodiments of the invention are contemplated and are within the spirit and scope of the invention. For example, phenylephrine or oxymetazoline can be selected as alternative vasoconstrictors in Examples 1 and 2. Thrombin can be selected as an alternative procoagulant in the compound of Example 3, as can an acetylglucosamine polysaccharide or a starch derived from potato. The foregoing examples and embodiments thus are not intended to be exhaustive, rather they are meant to be illustrative.

Experimental Test Results

A series of tests of several versions of the present invention were conducted to determine safety, composition, and hemostatic efficacy. All tests were performed with informed consent on healthy males between the ages of 50 and 60, who were also anti-coagulated using aspirin, herbal preparations, Plavix or a combination of these anti-coagulants. (Those of skill in the art will appreciate that the anti-coagulants were used to promote bleeding to facilitate observation and recordation of the bleeding inhibition effect, if any, of the invented compound.)

Because systemic application of epinephrine can potentially have profound effects including bronchial smooth muscle relaxation, cardiac stimulation, vasodilation in skeletal muscle, stimulation of glycogenolysis in the liver and so on, the first safety test involved topical application of one-half milliliter of a 10% epinephrine solution to the subject's unbroken skin to determine if any respiratory or hemodynamic symptoms followed. No symptoms were detected. A second safety test was conducted using an alcohol-epinephrine solution, wherein two milliliters were topically applied to each of two subjects' unbroken skin, while monitoring electrocardiogram and blood pressure. No abnormal signs or symptoms were detected. It was during this series of tests that a requirement for a higher viscosity was detected.

Subsequent series of tests were performed to determine hemostatic efficacy of an alcohol gel in which various concentrations of epinephrine were applied to the volar aspects of the test subjects' forearms in the areas of small bleeding punctures and comparing the bleeding times with similar applications of an alcohol gel without epinephrine. Presence or absence of bleeding was determined at 30-second intervals following the puncture by blotting the puncture with white tissue and visually observing whether blood was present. The gels were applied 30 seconds after each puncture. A first series showed that epinephrine concentrations of substantially less than 4% were inadequate to conclusively demonstrate a significant decrease in bleeding time.

A second series of similar tests of the invented compound was conducted, but with a version having an epinephrine concentration of approximately 5%, where the first application to a puncture used a gel without epinephrine; this was followed by a second application to a separate puncture using a gel containing an approximately 5% epinephrine concentration. Bleeding times for the gel without epinephrine averaged approximately 270 seconds. Bleeding times for the gel containing epinephrine averaged approximately 120 seconds for a bleeding time reduction of approximately 56%. Subsequent observations of the puncture sites revealed no unusual sequelae or re-bleeds.

Similar clotting time improvements were observed in a test series employing granulated chitosan suspended in an alcohol gel, wherein the chitosan comprised approximately 30% by volume of the gel formulation.

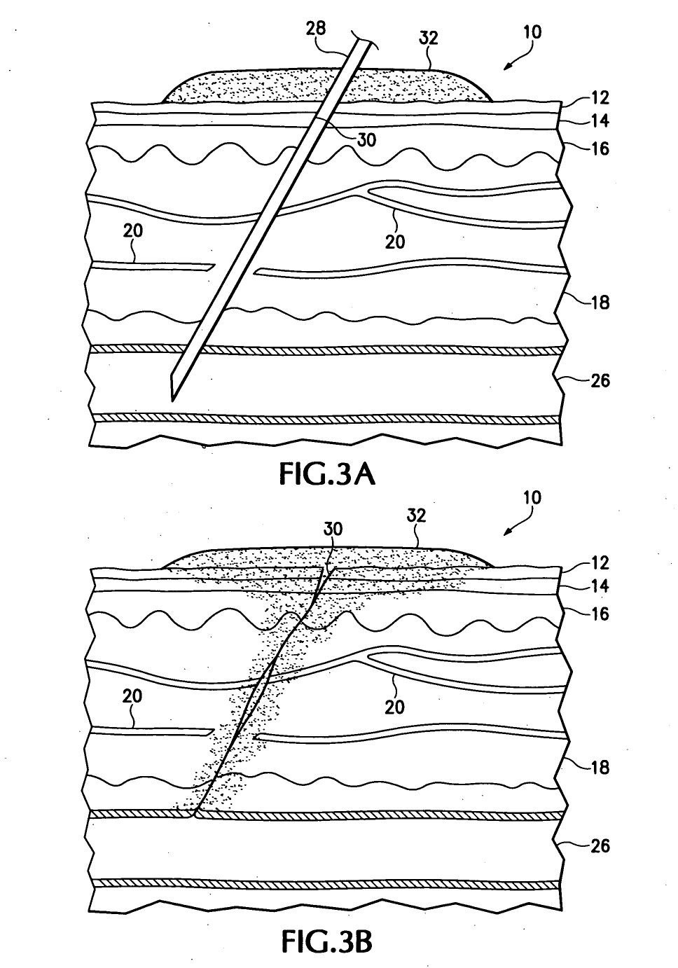

FIG. 2 is a flowchart that illustrates the method of use 200 of the compound in accordance with one embodiment of the invention. Use method 200 includes a) at 202, commencing cannulation sheath withdrawal; b) at 204, starting application of a hemostatic compound such as disclosed and claimed herein; c) at 206, withdrawing the cannulation sheath; and at 208, ceasing application of the invented hemostatic compound. This is further illustrated in FIGS. 3A-3C, discussed below. The method can further include e) at 210, commencing application of manual compression; and f) at 212, ceasing application of manual compression. It will be appreciated that steps 204 and 210 preferably commence at approximately the same time. It will also be appreciated that steps 206 and 212 typically terminate at different times, e.g. manual compression typically continues for seconds or even minutes after application of the hemostatic compound terminates. Furthermore, the timing of the various steps is illustrative only and is not intended to limit the invented method in any way, except when expressly so limited in the claims.

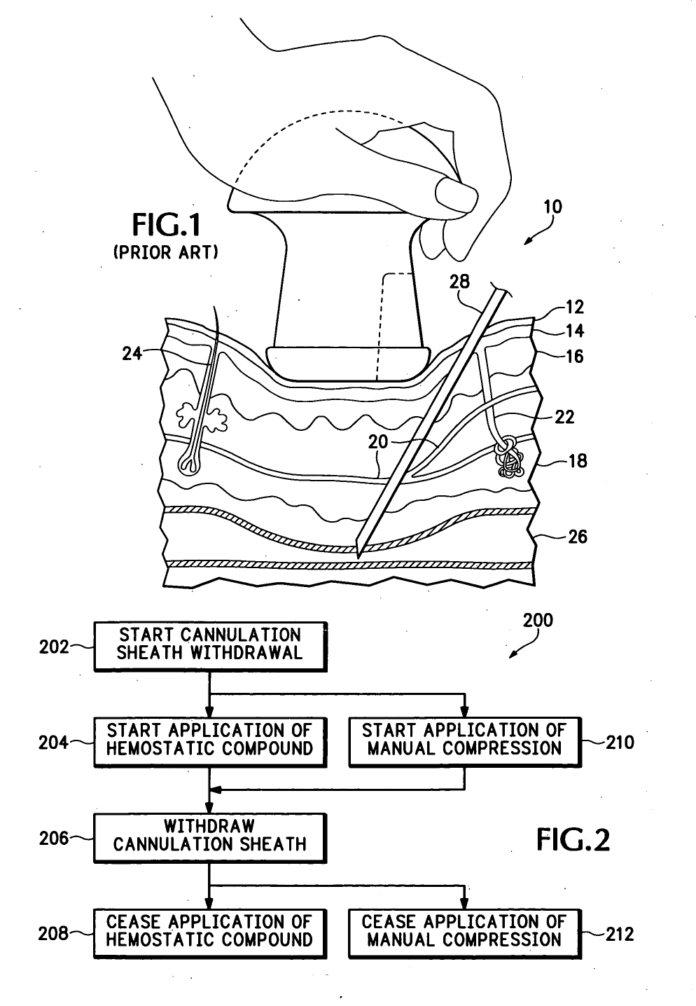

In accordance with one embodiment of the invention, the compression steps are manual and are unassisted by a compression device. In accordance with another embodiment of the invention, compression is manual and is assisted by use of a compression device such as that shown in FIG. 1, as discussed above. If unassisted, it will be understood that direct hand or finger pressure often aided by pressure from the other hand of the clinician is envisioned. If assisted, it will be understood that pressure from the palm of the clinician to the femoral artery or other wound site is leveraged to great mechanical advantage, i.e. it is focused and facilitated and made more convenient and comfortable. Those of skill in the art finally will appreciate that use of the hemostatic compound in accordance with the invention can mitigate the need for assisted or unassisted manual compression, although the two are thought to complement each other nicely. In any event, use of the invented compound relaxes the time and effort required to stop a post-catheterization or other cannulation procedure.

Assisted manual compression can be enhanced by the use of the patented and/or patent-pending Compressar® or Compass™ products mentioned above. Such are available from Advanced Vascular Dynamics of Portland, Oreg., USA.

Other steps not shown can also be performed, in accordance with the invented method, or the same steps can be performed somewhat differently or with somewhat different timing, so long as they accomplish the intended purpose, which is to inhibit bleeding at a wound site produced by cannulation.

FIG. 3A (which is somewhat simplified (for example, no hair follicle or sweat gland is shown) and which is not drawn to scale, for purposes of legibility) in fragmentary cross section shows a mammal's skin at 10 with a cannula or cannular sheath 28 extending therethrough and into an outer wall of a femoral artery 26. Cannula 28 extends within a cannulation track 30 produced by the forced introduction of cannula 28 into skin 10. It can be seen that the force of the introduction of cannula 28 and the sharpness of the tip of cannula 28 has produced undesirable but unavoidable trauma (e.g. openings and/or vivisections) within microcirculation system 20 (e.g. small vessels) of dermis 16.

FIG. 3A shows invented compound 32 applied topically to the exposed, outer surface of skin 10 (e.g. on the stratum corneum 12 thereof). By virtue of its unique properties, compound 32 does not flow, fall or drip from the area of its application. Instead it forms a blob or, liquid or semi-liquid film over a region surrounding the cannular puncture wound, as shown. Those of skill in the art will appreciate that FIG. 3A shows compound 32 immediately after its topical application, before evaporation of its evaporative agent into the air and before migration of its hemostatic agent through stratum corneum 12 and viable epidermis 14 and into dermis 16 and somewhat therebeyond, around the puncture wound site. This helpful viscosity characteristic of invented compound 32 is discussed further elsewhere herein.

FIG. 3B shows invented compound 32 in a slightly later phase of the invented method immediately following removal of cannula 28 from skin 10. Cannulation track 30 can be seen to have closed on itself to a great extent, along the line of the puncture wound. Invented compound 32 can be seen already to have produced inward migration of the hemostatic agent contained therein through stratum corneum 12 and through viable dermis 14, as indicated by stippling. Those of skill in the art will appreciate that such migration is enhanced by inclusion in compound 32 of not only a hemostatic agent but also a migration-enhancing agent. The liquid or semi-liquid component of invented compound 32 can be seen already to have commenced evaporation from the surface of skin 10. Those of skill in the art also will appreciate that such evaporation is desirable, in that it leaves the surface of the skin proximate the wound site relatively quickly dry to the touch and to environmental contaminants, e.g. airborne particulates, and enables easier subsequent manipulation and dressing of such site.

The modality of the bleeding inhibition thus involves enabling a hemostatic agent that is applied topically to the exterior surface of a mammal's skin nevertheless to migrate effectively inwardly therefrom across the substantial barriers produced by the stratum corneum to underlying tissue.

After application of the invented compound illustrated in FIGS. 3A and 3B (described above), it will be understood that further evaporation of the liquid or semi-liquid components of compound 32 as well as further migration of the hemostatic agent occurs. Typically, evaporation continues for several seconds up to a minute or more after removal of cannula 28 from skin 10. Nevertheless, a thin residue of compound 32 remains on the outer surface of the patient's skin, the residue possibly still containing a residual dose of the hemostatic agent.

Those of skill will appreciate that use of the invented compound is not limited to hospital or clinical settings, or to any particular medical procedure, e.g. cannulation. Indeed, the invented compound finds broad utility in field and home uses such as so-called ‘first aid’ treatment following minor or serious accidental injury such as puncture, incision or abrasion that might occur at home, in the workplace or anywhere. Such first aid or emergency uses and their efficacy in bleeding stoppage are illustrated above in relation to the described experimental test results. Thus, many uses of the invented compound, as well as alternative methods of its use, are contemplated as being within the spirit and scope of the invention.

It will be understood that the present invention is not limited to the method or detail of construction, fabrication, material, application or use described and illustrated herein. Indeed, any suitable variation of fabrication, use, or application is contemplated as an alternative embodiment, and thus is within the spirit and scope, of the invention.

From the foregoing, those of skill in the art will appreciate that several advantages of the present invention include the following.

The present invention provides an inexpensive, non-invasive, quick and relatively clean mechanism to stop bleeding at a puncture, incision, abrasion or other wound site. The invented compound is applied topically, i.e. to the external surface of the skin, but because of its unique components it is effective to promote hemostasis where needed beneath the skin's surface by delivering a hemostatic agent to the tissue underlying the stratum corneum. The delivery is enabled and/or facilitated by a transdermal migration-enhancing agent and via migration down the cannulation track.

The compound can have any or all added agents, including an astringent agent, a sanitizing agent, a sterilizing agent, an analgesic agent, a skin permeability agent, an antibiotic agent, a skin adhesive agent, an evaporative agent, an aloe agent, a tocopherol agent, a fragrance agent and a cosmetic agent. The compound can be applied with any suitable applicator, e.g. a swab or tissue, or without such applicator, for example, by using a squeeze tube or bottle. The compound finds particular utility in connection with catheterizations that are performed in hospital settings or in the event of accidental punctures, incisions and abrasions that can occur anywhere.

Analgesic agents reduce pain. Skin adhesive agents, e.g. liquid suture materials or other gluey substances, promote skin adherence and wound closure. Aloe agents promote healing. Tocopherol agents such as vitamin E promote skin regeneration. Fragrance agents provide more pleasing bouquets. Cosmetic agents such as foundations, bronzers, blot powders, blushers, cover-ups, etc. cover bruises, blemishes or other visible skin trauma.

Advantages of the invention include the ability to easily and non-invasively apply a hemostatic and evaporative gel or transdermal migration-enhancing or sanitizing agent, optionally with a microbicidal and other agents as described herein, to help stop bleeding and prevent infection at a wound site. The ability to apply an otherwise liquid, hard-to-control substance, in the form of an easy-to-control substance which substantially evaporates after application, results in a convenience benefit and targeted-site-application benefit. The ability to apply all of said substances at once, to save time, is a great advantage. Unintended and/or undesirable systemic effects nevertheless are avoided, thus rendering the compound's use safe even for vulnerable patients, i.e. patients with pre-existing conditions such as dysrhythmias.

It is further intended that any other embodiments of the present invention that result from any changes in application or method of use or operation, method of manufacture, shape, size, or material which are not specified within the detailed written description or illustrations contained herein yet are considered apparent or obvious to one skilled in the art are within the scope of the present invention.

Accordingly, while the present invention has been shown and described with reference to the foregoing embodiments of the invented compound and its use, it will be apparent to those skilled in the art that other changes in form and detail may be made therein without departing from the spirit and scope of the invention as defined in the appended claims.

1-22. (canceled)

23. A topical medicinal compound comprising;
a hemostatic agent for inhibiting vascular bleeding; and
an evaporative gelling agent for imbuing the compound with viscosity properties that substantially retain the compound within the skin surface area where the compound is applied for a defined period of time after topical application before substantially evaporating;
the agents combined in a defined ratio to a liquid or semi-liquid form capable of topical application to an abrasion, puncture or incision site and capable at the site of inhibiting bleeding.

24. The compound of claim 23, wherein the hemostatic agent includes one or more of a vasoconstrictor and a procoagulant.

25. The compound of claim 23, wherein the evaporative gelling agent includes at least an alcohol as an evaporative agent and one or more high molecular weight cross-linked polymers of acrylic acid as a gelling agent.

26-31. (canceled)

32. The compound of claim 23, wherein the liquid or semi-liquid form produced by the defined ratio is characterized by viscosity properties of between approximately 25,000 and 250,000 centipoise.

33. The compound of claim 25, wherein the alcohol is present within the compound in a volume ratio of between approximately 20% and 90%.

34. The compound of claim 33, wherein the alcohol is present within the compound in a volume ratio of between approximately 40% and 80%.

35. A topical medicinal compound comprising:
a hemostatic agent for inhibiting vascular bleeding; and
a flowable evaporative gelling agent for imbuing the compound with viscosity properties that retain the compound within the skin surface area where the compound is applied for a brief period of time after topical application and for further imbuing the compound with evaporative properties that cause a portion of the compound to evaporate relatively rapidly thereafter;
the agents combined in a defined ratio in a form capable of topical application to an abrasion, puncture or incision site and capable at the site of inhibiting bleeding.

36. The compound of claim 35, wherein, per milliliter volume of the compound, the brief period of time before evaporation is less than approximately 300 seconds.

37. The compound of claim 36, wherein, per milliliter volume of the compound, the brief period of time before evaporation is less than approximately 60 seconds.

38. The compound of claim 35, wherein the evaporative gelling agent includes an alcohol in a greater than approximately 20% concentration by volume of the compound.

39. The compound of claim 38, wherein the evaporative gelling agent includes an alcohol in a greater than approximately 40% concentration by volume of the compound.
